(19) United States
(12) Patent Application Publication
(10) Pub. No.: US 2005/0075672 A1
(43) Pub. Date: Apr. 7, 2005

(54) Cardiac Stimulation Apparatus With Multiple Input Sense Amplifiers

(76) Inventor: William B. Rottenberg, Durango, CO (US)

Correspondence Address: JOHN RICHARD MERKLING, 11171 WEST EXPOSITION DRIVE, LAKEWOOD, CO, 80226-3867, US

(21) Appl. No.: 10/605,525

(22) Filed: Oct. 6, 2003

(51) Int. Cl. A61N 1/362

(52) U.S. Cl. 607/9

(57) ABSTRACT

An implantable cardiac stimulation system having a cardiac stimulator having a multi-electrode lead attached to the stimulator. The electrodes may be circumferential coils or rings. Each one of one or more sets of electrodes, each set of electrodes comprising a plurality of electrodes, is associated with a single sense amplifier. Switches sequentially connect the sense amplifier to each of the electrodes in the attached set. A switching or sampling rate is maintained such that significant information regarding the electrical condition of the heart can be extracted. Switches connect inactive feedback capacitors to ground thereby maintaining the band pass characteristics of a selected channel.

### BACKGROUND OF INVENTION

This invention pertains to a method and apparatus for applying cardiac stimulation using multiple electrodes, and more particularly, to a method and apparatus for employing a single operational amplifier to sense on multiple electrodes.

The heart is a mechanical pump that is stimulated by electrical impulses. The mechanical action of the heart results in the flow of blood. During a normal heartbeat, the right atrium fills with blood from the returning veins. The right atrium then contracts and the blood moves into the right ventricle. When the right ventricle contracts, it pumps blood to the lungs. Blood returning from the lungs moves into the left atrium, and from the left atrium, it moves into the left ventricle. The left ventricle pumps blood throughout the body. Four heart valves keep the blood flowing in the proper directions.

The electrical signal that drives this mechanical contraction starts in the sinus node, a collection of specialized heart cells in the right atrium that automatically depolarize (change their voltage potential). This depolarization wave front passes across all the cells of both atria and results in atrial contraction. When the advancing wave front reaches the A-V node it is delayed so that the contracting atria have time to fill the ventricles. The depolarizing wave front then passes over the ventricles, causing them to contract and pump blood to the lungs and body. This electrical activity occurs approximately seventy-two times a minute in a normal individual and is called normal sinus rhythm.

The corresponding electrical signals identifying these events are usually referred to as the P, QRS (or R) and T waves. More particularly, an atrial contraction is represented on an ECG by a P wave, a ventricular contraction is represented by an R wave and a ventricular repolarization is represented by a T wave. The atrium also repolarizes, but this event (the U wave) is masked by activity in the ventricle and consequently it is not observable on an ECG.

Conventional pacemakers utilize single or dual electrode leads to apply pacing pulses. The dual electrode (bipolar) lead typically includes a tip and a ring electrode. The lead is inserted in such a manner that the tip is imbedded into the cardiac muscle. A pacing pulse is then applied between the tip and the ring electrodes, thereby causing the cardiac muscle to contract. If a single unipolar electrode lead is used, the electric pulse is applied between the tip electrode and another electrode outside the heart, for example, the housing of the pacemaker. Bradycardia pacing therapy has usually been delivered through a pacing electrode implanted near the ventricular apex, that is, near the bottom of the heart. This location has been preferred not for physiologic reasons, but because most lead designs favor implantation at this site. A lead entering the right ventricle from the right atrium tends to extend into the lower apex of the ventricle where an active fixation apparatus, such as a helical corkscrew, may be used to secure the lead to the heart wall. Even if the distal tip of the lead is implanted at another location, it may be difficult or impossible to move the electrode to another location within the heart after initial implantation.

Multiple stimulating electrodes may permit an implantable pacemaker to stimulate close enough to a physiologically preferred location in the patient's heart to cause improved cardiac efficiency. Moreover, an apparatus with a single electrode cannot control cardiac contraction, guide the propagation of a wave front, force a selected path for a stimulating wave front, or create a coordinated simultaneous or near simultaneous cardiac contraction of large sections of the myocardium. Such controlled contractions may result in more efficient cardiac contraction, thereby reducing the overall demand on the heart, allowing the body to alleviate the symptoms associated with inefficient blood flow.

Sensing on multiple electrodes may also allow more accurate and complete diagnosis of the condition of the heart. The direction and speed of wave fronts may be detected as well as the origins of contractions or other phenomena. Sensing at each electrode through an operational amplifier dedicated solely to that electrode is, however, energy expensive. In an implantable device where longevity is limited by energy consumption and battery size, it is important to reduce energy use as much as possible.

### SUMMARY OF INVENTION

In view of the above disadvantages of the prior art, it is an objective of the present invention to provide an implantable cardiac stimulation system, such as a pacemaker, in which three or more electrodes are positioned in a chamber of the heart and multiple sensing electrodes are used with a single sense amplifier.

A further objective is to provide an implantable cardiac stimulation system with multiple sense amplifiers, each amplifier serving a plurality of sense electrodes.

Another object of the invention is to provide a sense amplifier in a cardiac stimulation system that uses multiple electrodes and that sequentially switch through any or all of the electrodes.

A further object of the invention is to provide a sense amplifier in a multiple-electrode cardiac stimulation system that maintains band pass characteristics while switching between electrodes.

Other objectives and advantages of the invention shall become apparent from the following description.

Briefly, the subject invention pertains to an implantable cardiac stimulation system having a cardiac stimulator having electronic circuitry for the stimulation and a multi-electrode lead attached to the stimulator and inserted into one or more body cavities. (The term cardiac stimulator will be used herein to cover pacemakers as well as other cardiac devices such as internal cardioversion devices and defibrillators.) The lead is inserted into the cardiac cavity into a predetermined position. Alternatively the lead may be positioned in the veins, or it may be positioned externally of the heart.

In a preferred embodiment, a lead having an elongated member is provided with the electrodes being formed on said elongated member. The electrodes comprise axially spaced electrodes disposed on said elongated member, each electrode being connected by a wire extending though said elongated member. The electrodes may be circumferential coils integral or continuous with the wires or may be rings connected to the wires by crimping or laser welding, for example. An electrode may also be provided at the distal end of the lead. The elongated member may be a tube housing the wires. The electrodes can be angularly spaced with respect to each about the elongated member.

Each one of one or more sets of electrodes, each set of electrodes comprising a plurality of electrodes, is associated with a sense amplifier. Switches sequentially connect the sense amplifier to each of the electrodes in the attached set. A switching or sampling rate is maintained such that significant information regarding the electrical condition of the heart can be extracted. Switches connect inactive feedback capacitors to ground thereby maintaining the band pass characteristics of a selected channel.

### BRIEF DESCRIPTION OF DRAWINGS

FIG. 1 shows a diagrammatic front view of a patient with a cardiac stimulation system.

FIG. 2 shows a block diagram of the cardiac stimulator of FIG. 1.

FIG. 3 is a block diagram of a portion of the circuits of FIG. 2.

FIG. 4 is a second embodiment of the circuit portion of FIG. 3.

FIG. 5 is a block diagram of another portion of the circuits of FIG. 2.

FIG. 6 is a diagram of a first embodiment of a multi-input amplifier.

FIG. 7 is a diagram of a second embodiment of a multi-input amplifier.

FIG. 8 is a timing diagram for controlling switches in the multi-input amplifiers of FIGS. 6 and 7.

FIG. 9 is a timing diagram distinguishing the timing of an output switch.

FIG. 10 is a timing diagram showing exemplary input and output waveforms.

FIG. 11 is a view of a multi-electrode lead implanted in a heart.

FIG. 12 is a plan view of a coil electrode.

FIG. 13 is a cross sectional plan view of a ring electrode.

FIG. 14 is a cross section of the multi-electrode lead of FIG. 11, taken along line 14-14 in FIG. 12.

### DETAILED DESCRIPTION

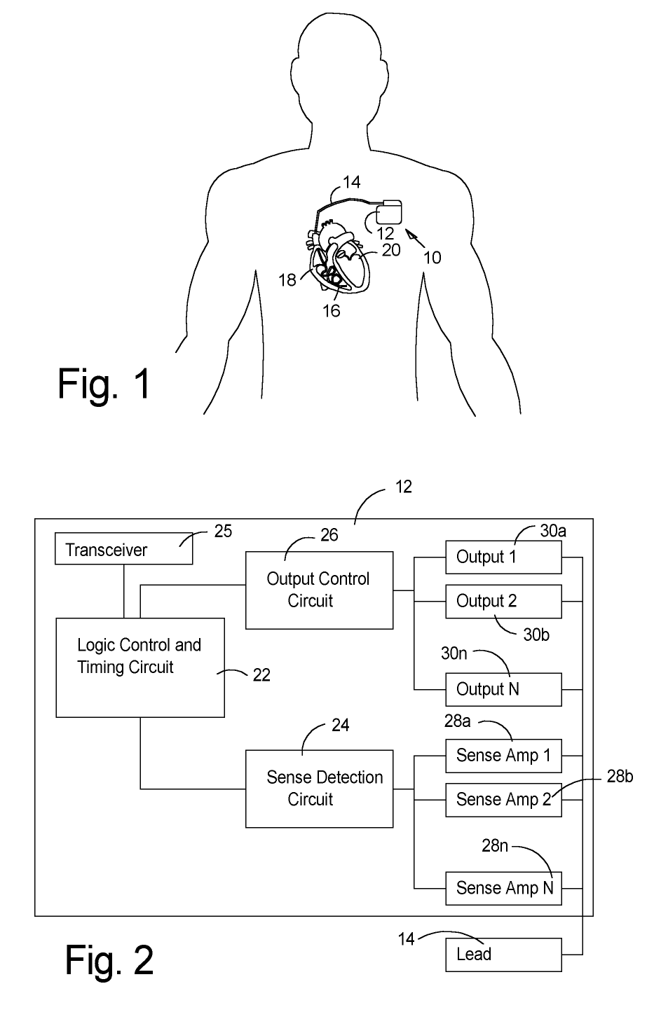

The subject invention pertains to an implantable cardiac stimulation system 10 including a cardiac stimulator 12 with various electronic circuits, and a multi-electrode lead 14 attached to the stimulator 12, as shown in FIG. 1. The lead 14 has a distal end 16 disposed, for example, in one of the cardiac chambers such as the right ventricle 18 of heart 20. In FIG. 1, end 16 is shown having a general spiral shape. The system 10 is adapted to deliver therapy in the form of electrical pulses. The therapy may include GCV (greater cardiac vein) resynchronization therapy, treatment of conduction pathway abnormalities, bardycardia pacing, etc. The cardiac stimulator 12 contains electronic components common to current cardiac stimulators such as a battery, microprocessor control circuit, ROM, RAM, an oscillator, reed switch and antenna for communication, and output circuits. Types of these components are well known to those of skill in the art. In addition, the cardiac stimulator 12 has a plurality of independent sensing and stimulating circuits for each heart chamber, as will be explained below, and, particularly, at least one sensing circuit using a single operational amplifier for multiple electrodes.

Cardiac Stimulator

FIG. 2 illustrates important elements of the cardiac stimulator 12 in block diagram. The cardiac stimulator 12 comprises a logic control and timing circuit 22, which may include a microprocessor and memory, but which could also be implemented in a specialized circuit. The logic control and timing circuit 22 receives input from a sense detection circuit 24 and issues control instructions to an output control circuit 26. To accommodate the many electrodes used in the apparatus, multiple sense amplifiers 28a, 28b . . . 28n may be provided, each amplifier in electrical communication with multiple electrodes (not shown in this view) through the lead 14 and with the sense detection circuit 24, as will be explained in greater detail below. Similarly, the output control circuit 26 is electrically connected to a plurality of output circuits 30a, 30b . . . 30n. The output circuits 30a, 30b . . . 30n produce stimulating pulses or high frequency, non-simulating signals at electrodes in the heart through the lead 14. The logic control and timing circuit 22 may operate in accordance with a program stored into memory. Programming instructions are received through a transceiver 25, for example from an external programmer (not shown). The sensing detection circuit 24 senses intrinsic activity and other signals within the heart 20 and provides corresponding indication signals to the microprocessor. The logic control and timing circuit 22 then issues appropriate commands to the output control circuit 26. The output control circuit 26 generates appropriate stimulation pulses. These pulses are steered to a selected electrode or electrodes.

Output Circuits

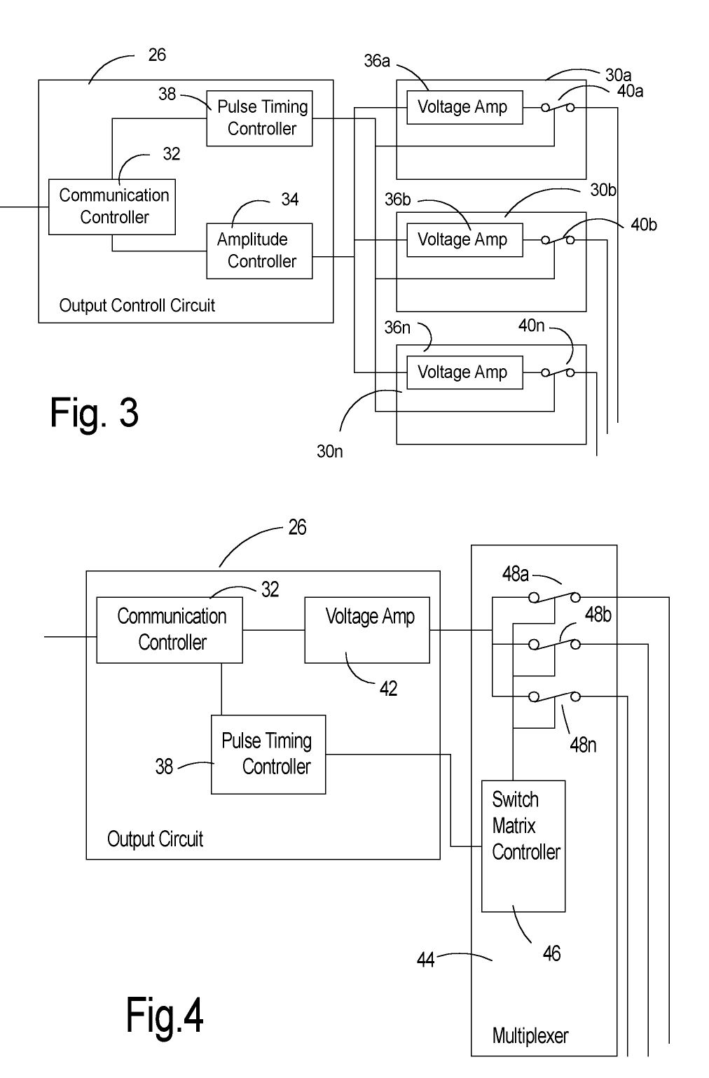

FIGS. 3 and 4 show two embodiments of output control circuits 26 and output circuits 30a, 30b . . . 30n. The embodiment of FIG. 3 comprises a communications controller that receives control signals from the logic control and timing circuit 22 (FIG. 2). Output of the communications controller 32 is sent to an amplitude controller 34 that controls the voltages produced by a plurality of voltage amplifiers 36a, 36b . . . 36n. In parallel, the communications controller 32 also regulates a pulse timing controller 38. Signals from the pulse timing controller 38 close and open switches 40a, 40b . . . 40n, thereby delivering stimulation pulses or high frequency signals to the heart through electrodes on the lead 14.

The embodiment of FIG. 4 also uses a communication controller 32 and pulse timing controller 38, but the amplitude controller 34 and plurality of voltage amplifiers 36a, 36b . . . 36n are replaced by a single voltage amplifier 42. To achieve the same effect of multiple pulses to selected electrodes, the signals from the pulse timing controller are sent to a multiplexer 44, comprising a switch matrix controller 46 and a plurality of switches 48a, 48b . . . 48n. The switches 48a, 48b . . . 48n must be opened and closed in a synchronized manner. The embodiment of FIG. 4 gains energy efficiency by minimizing the number of voltage amplifiers.

Sense Circuits

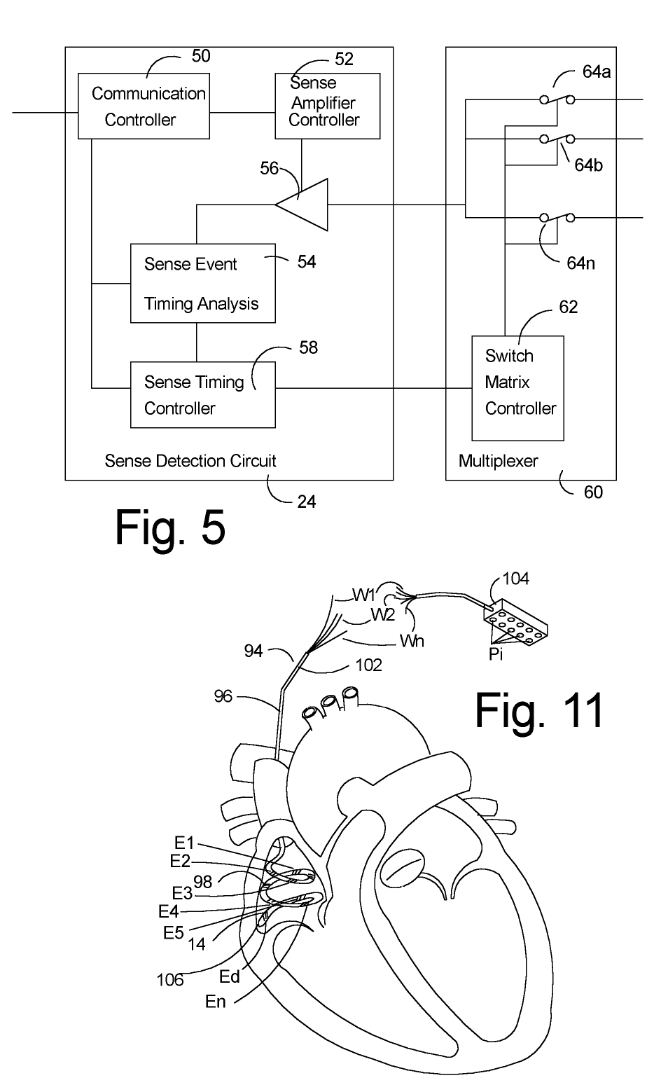

A variety of apparatus may also be used to sense signals from multiple electrodes through the sense detection circuit 24. A sense circuit illustrated in FIG. 5 employs a multiplexer in a manner similar to the second embodiment of the output control circuit, described in connection with FIG. 4, above. In the sense detection circuit 24, a sense amp controller 52 controls a single amplifier 56 connected to multiple electrodes. As shown in FIG. 2, multiple amplifiers may be provided in a single device, each amplifier being connected to multiple electrodes. Thus, for example, each amplifier may be connected to four electrodes and eight amplifiers may service thirty-two sense electrodes. The sense event timing analysis unit 54 analyses the output of the single amplifier 56 and produces an output corresponding to a moving wave front. A sense timing controller 58, in electrical communication with both the communication controller 50 and the sense event timing analysis unit 54, controls a multiplexer 60 through a switch matrix controller 62. The switch matrix controller 62 opens and closes a plurality of switches 64a, 64b . . . 64n, selectively connecting the electrodes of the lead 14 to the sense amplifier 56. As explained above, replacing multiple dedicated sense amplifiers 36a, 36b . . . 36n with a single amplifier 56 exchanges flexibility and simplified control for energy efficiency. In an implantable device such as a cardiac stimulator, energy conservation can be of paramount importance. Even low power amplifiers consume one to two μA. A circuit comprising four amplifiers, for example, might use four to eight μA for sensing amplifiers alone. A sense amplifier that can service multiple channels, as described below, can greatly benefit an implantable multi-electrode device.

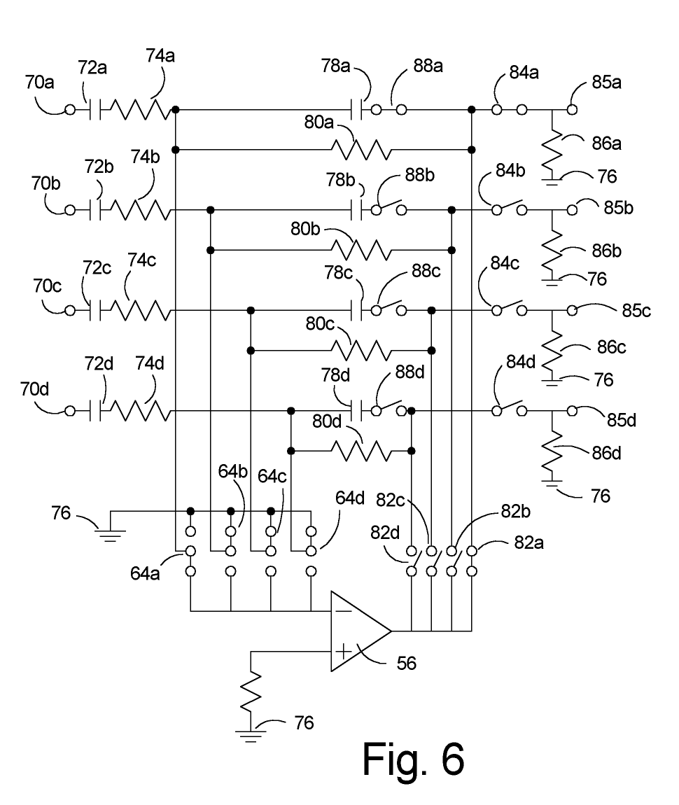

A first embodiment of a multi-channel sense amplifier is illustrated in FIG. 6. Multiple input lines 70a, 70b, 70c, 70d may be connected through the lead to electrodes on the lead 14. Each input line has a filter capacitor 72a, 72b, 72c, 72d and resistor 74a, 74b, 74c, 74d. It is expected that wherever resistors are indicated herein, such components may be implemented it any suitable manner, including, preferably, by means of switched capacitors, as is well known in the art. Switched capacitors are relatively easily implemented in integrated circuitry and have been used in implantable medical devices heretofore. Each resistor (or switched capacitor resistance element) 74a, 74b, 74c, 74d connects to a double throw switch 64a, 64b, 64c, 64d. When the multi-channel sense amplifier senses through a particular input line, for example input line 70a, the switch 64a for that line connects to the negative input of the amplifier 56. Simultaneously, each of the other switches 64b, 64c, 64d connect their respective lines to system ground 76. Grounding the unused input lines is important to maintain the frequency response for each channel. Without grounding the input, the frequency response of each filter changes when the channel is not selected and the frequency cutoff for the channel changes.

Each input line or channel also has an associated feedback capacitor 78a, 78b, 78c, 78d and feedback resistor 80a, 80b, 80c, 80d. When the multi-channel amplifier senses through a particular input line, for example input line 70a, a feedback switch 82a connects the output of the amplifier 56 back through the feedback capacitor 78a and feedback resistor 80a to the input of the amplifier 56. Simultaneously, feedback switches 82b, 82c, 82d for each of the other input lines 70b, 70c, 70d are open, disconnecting these paths from the circuit. Output switches 84a, 84b, 84c, 84d connect the amplifier output to the rest of the circuit as shown in FIG. 5. When sensing on a particular line, for example input line 70a, output switch 84a closes, while the other output switches 84b, 84c 84d open. Output resistors 86a, 86b, 86c, 86d connect the output sides of the output switches 84a, 84b, 84c, 84d to system ground 76 and allow the output to return to ground value when the channel is not being sampled.

Capacitor hold switches 88a, 88b, 88c, 88d are connected in series with the feedback capacitors 78a, 78b, 78c, 78d. The capacitor hold switches 88a, 88b, 88c, 88d prevent their associated feedback capacitor 78a, 78b, 78c, 78d from discharging through an associated feedback resistor 80a, 80b, 80c, 80d. For example, if sensing is taking place through input line 70a, capacitor hold switch 88a is closed and the remaining capacitor hold switches 80b, 80c, 80d are open. In this embodiment, the high pass poles of each of the channels is maintained but the low pass pole may shift slightly because part of the circuit is disabled when a channel is not selected.

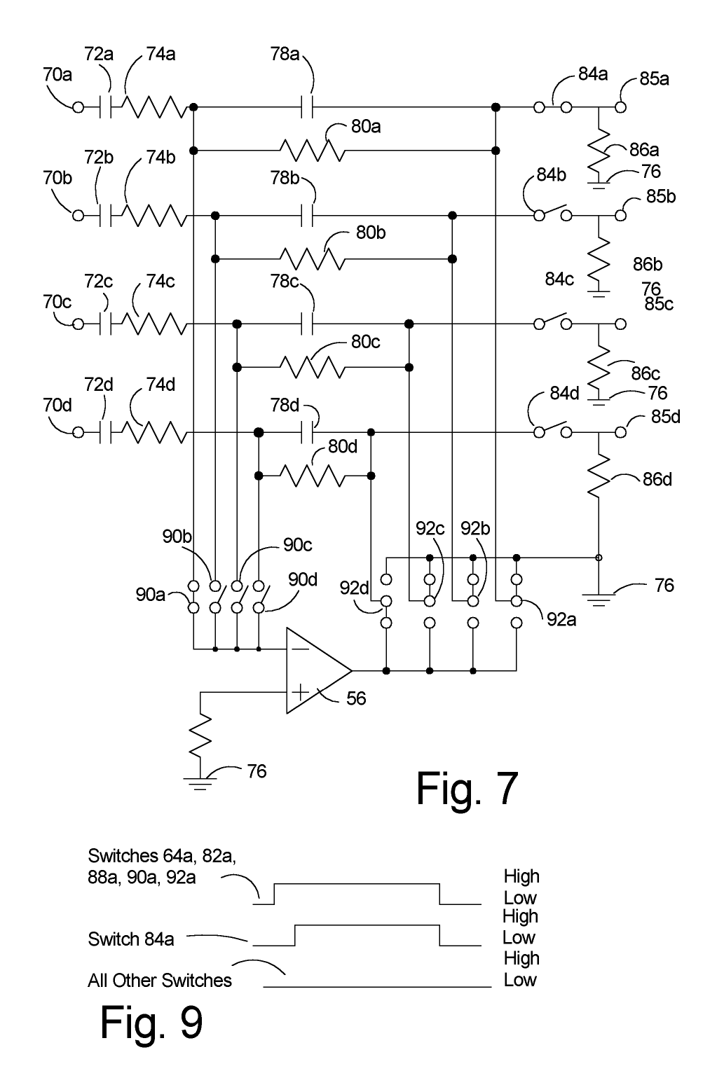

In a second embodiment, illustrated in FIG. 7, both the high pass poles and the low pass poles move slightly, but the low pass pole moves less than in the first embodiment of FIG. 6. As on the embodiment of FIG. 6, multiple input lines 70a, 70b, 70c, 70d may be connected through the lead to electrodes on the lead 14. Each input line has a filter capacitor 72a, 72b, 72c, 72d and resistor 74a, 74b, 74c, 74d. Each resistor 74a, 74b, 74c, 74d connects to a single throw switch 90a, 90b, 90c, 90d. When the multi-channel sense amplifier senses through a particular input line, for example input line 70a, the switch 90a for that line connects to the negative input of the amplifier 56. Simultaneously, each of the other switches 90b, 90c, 90d are opened.

Each input line or channel also has an associated feedback capacitor 78a, 78b, 78c, 78d and feedback resistor 80a, 80b, 80c, 80d. When the multi-channel amplifier senses through a particular input line, for example input line 70a, a double throw feedback switch 92a connects the output of the amplifier 56 back through the feedback capacitor 78a and feedback resistor 80a to the input of the amplifier 56. Simultaneously, feedback switches 92b, 92c, 92d for each of the other input lines 70b, 70c, 70d connect their respective lines to system ground 76. Grounding the unused input lines is important to maintain the frequency response for each channel. Without grounding the input, the frequency response of each filter changes when the channel is not selected and the frequency cutoff for the channel changes. Output switches 84a, 84b, 84c, 84d connect the amplifier output to the rest of the circuit as shown in FIG. 5. When sensing on a particular line, for example input line 70a, output switch 84a closes, while the other output switches 84b, 84c 84d open. Output resistors 86a, 86b, 86c, 86d connect the output sides of the output switches 84a, 84b, 84c, 84d to system ground 76 and allow the output to return to ground value when the channel is not being sampled.

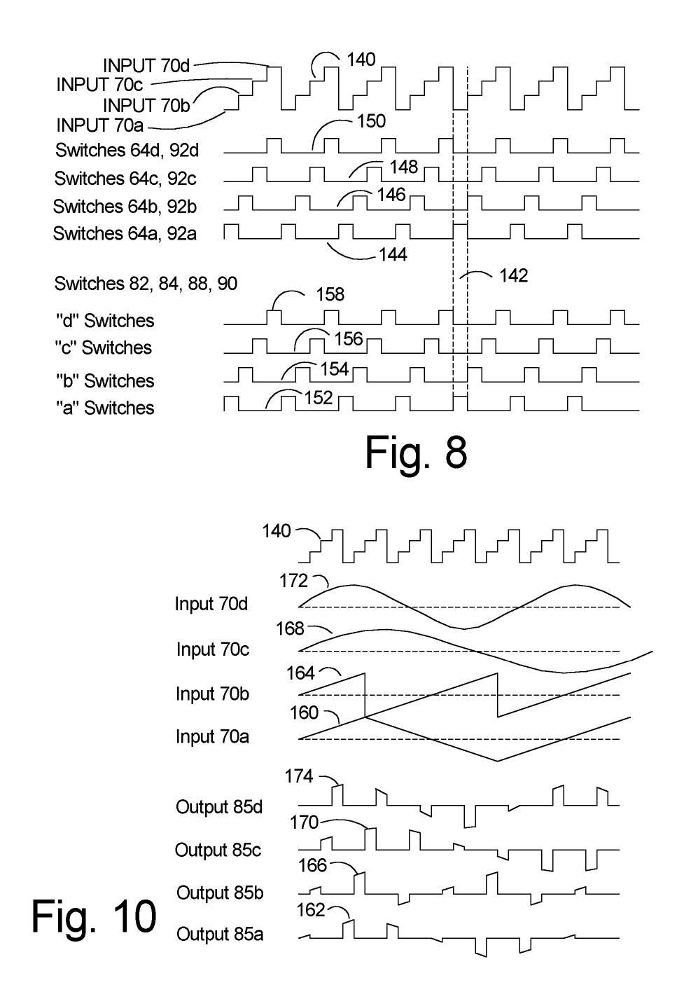

FIG. 8 is a graphic representation of the timing sequence of the switches. The upper line represents the selection of each input line or channel 70a, 70b, 70c, 70d from which a signal from an electrode will be sensed. In this example, the first input line 70a or channel is selected first, the second line 70b is selected next, followed by the third line 70c and finally the fourth line 70d. It should be understood that any number of input lines may be connected through a single amplifier, limited only by switching rates and the ability of the circuit to acquire a meaningful signal, as will be discussed below. In addition, a single implantable device could have multiple amplifiers, each amplifier connected through a switch network as described above to a plurality of electrodes.

Each of the two embodiments of FIGS. 6 and 7 has a bank of double pole switches with one pole connected to system ground. In the first embodiment of FIG. 6, these switches are the double throw switches 64a, 64b, 64c, and 64d connected to the input lines 70a, 70b, 70c, 70d respectively and to either ground 76 or to the inverting input of the amplifier 56. In the second embodiment of FIG. 7, the bank of double pole switches comprises the double throw feedback switches 92a, 92b, 92c and 92d. When a particular input line or channel is selected for sensing, the double throw switch associated with that line is connected to the amplifier (either the input or the output), while all other double throw switches are connected to ground. For example, if the first input line 70a is sensed, as indicted by a mark on the line 140 in the column 142, switch 64a or 92a is set to pass a signal, as shown by the line 144 in column 142. The other double throw switches 64b, 64c, and 64d or 92b, 92c and 92d connect to ground as indicated by the state of the lines 146, 148 and 150 in the column 142.

Each of the embodiments of FIGS. 6 and 7 has a set of four output switches 84a, 84b, 84c, 84d. The first embodiment of FIG. 6 has a set of four switches 82a, 82b, 82c, 82d on the output side of the amplifier 56. The second embodiment has a set of four switches 90a, 90b, 90c, 90d on the input side of the amplifier 56. Finally, the first embodiment has a set of four switches 88a, 88b, 88c, 88d. In each of these sets of switches, the a, b, c and d switches close or open at similar times. The state of the “a” switches is shown by line 152 in FIG. 8. The state of the “b” switches is shown by line 154. The state of the “c” switches is shown by line 156, and the state of the “d” switches, by line 158. The state of these switches provides an electrical path for sensing a particular line. For example, if the first input line 70a is sensed, as shown in column 142, the “a” switches 82a, 84a, 88a, 90a are on or closed, while the “b” switches 82b, 84b, 88b, 90b, the “c” switches 82c, 84c, 88c, 90c, and the “d” switches 82d, 84d, 88d, 90d are off or open. When the “b” switches close for the second input line 70b, the “a”, “c” and “d” switches are open. When the “c” switches close for the third input line 70c, the “a”, “b” and “d” switches are open. Finally, when the “d” switches close for the fourth input line 70d, the “a”, “b” and “c” switches are open. The four output switches 84a, 84b, 84c and 84d can be phased slightly with respect to the other switches represented by lines 152, 154, 156 and 158 to allow for settling time and to prevent noise or glitches from the other switches from being passed to the output. For the output switches, the duration of “on” or closed time should be shortened and should fall towards the end of the “on” period fro the other switches. This is illustrated in FIG. 9 for a single cycle for both circuits, corresponding to the state shown in column 142 of FIG. 8.

FIG. 10 shows a set of arbitrary inputs and outputs for four electrodes connected to four input lines, for example input lines 70a, 70b, 70c and 70d. The input waveforms are not intended to be cardiac waveforms. The distinctive shapes of the input waveforms have been selected to be more easily distinguished in the output waveforms. The line 140 in FIG. 10 corresponds to the same line 140 in FIG. 8 and shows sequential selection of input lines 70a, 70b, 70c and 70d for sampling. The input line 70a is shown carrying a voltage 160 comprising a series of alternating ascending and descending ramps. The corresponding output on line 85a is a series of amplified pulses defining points or segments of the voltage ramps. The input on line 70b is a saw-toothed waveform 164. The form of the input can be seen in the pulses 166 on line 85b. Similarly, the input on line 70c is represented as a sinusoidal waveform 168 while the input on line 70d is a second sinusoidal waveform 172 of a different period. The output pulses 170 on line 85c and the output pulses 174 on line 85d retain sufficient information to reconstruct the input waveforms. Each output waveform 162, 166, 170, 174 forms an envelope of the input signal. Additional filtering or signal processing can extract desired information such as frequency or relative amplitude. The resolution of the details of the input waveform depends on the scanning rate. A faster scanning rate usually produces a more detailed representation of the input signal. The scanning frequency is selected with regards to the number of channels served by a single amplifier and the high frequency cutoff or low pass pole of the amplifier. Nyquest's sampling theory states that, in order to determine the frequency of an input signal, the sampling rate must be at least twice the frequency of the signal. With a multi-channel system, the sampling rate must also be multiplied by the number of channels. Thus, if the highest expected frequency of input were 200 Hz, a sampling rate of at least 800 Hz would be necessary to adequately determine the frequency of the input wave forms. A higher sampling rate would allow discrimination of more detail of the input waveforms.

Multi-Electrode Lead

Details of the multi-electrode lead 14 are shown in FIG. 11. The lead 14 includes an external biocompatible polymer tube 94 having a straight portion 96 and a shaped portion 98. The tube may be made of polyurethane or other similar materials that may be thermally shaped so that the shaped portion 98 retains any desired configuration. In FIGS. 1 and 11, the shaped portion 98 is shown as having a spiral shape, but many other shapes may be selected as well to address the clinical needs of a particular patient.

A plurality of electrodes E1, E2, E3, E4, E5, . . . En are attached to tube 94 of the lead 14. Preferably electrodes E1 . . . En are formed of coils of bare wire or cable wound about the tube 94. Each electrode is connected to corresponding wires W1, W2, W3 . . . Wn which extend through the length of tube 94 and which are shown exiting through end 102 for the sake of clarity. Wires W1, W2, W3 . . . Wn are insulated, so that they are not shorted to each other within the tube 94. The electrode 14 and its method of manufacture are disclosed in co-pending commonly assigned U.S. application Ser. No. 09/245,246 filed Feb. 5,1999, and incorporated herein by reference. Preferably the end 102 of tube 94 and the ends of wires W1, W2, W3, etc. are coupled to a connector 104 for attaching the lead 14 to the cardiac stimulator 12. The connector 104 may have a plurality of pins Pi. Each wire W1 . . . Wn is associated with a pin. In addition to spiral coil or ring electrodes E1 . . . En, a distal tip electrode Ed may also be provided. The distal tip electrode Ed may also have an active fixation mechanism, for example a helical screw 106 or tines, to secure the lead to the interior wall of the heart.

The lead 14 can be constructed with the tube 104 extending relatively straight or can be customized to any shape to fit any pre-selected location within the heart 20 dependent on each particular patient's pathology. For example, if the lead 14 is to be placed in the greater cardiac vein, then its end 16 (consisting of shaped portion 98 and electrodes E1, E2, E3 . . . etc.) is shaped to form a small helix, so that it will fit into the greater cardiac vein.

The tube 94 can be formed with a longitudinal cavity 108, as shown in the cross sectional view of FIG. 14. Cavity 108 holds the wires W1, W2, W3 etc. The lead 14 could be straightened by inserting a substantially straight stylet 112 into an interior tube or lumen 114. The stylet 112 is also flexible but is less flexible than the lead 14 so that as it is inserted into the lumen 114, it forces the tube 94 to straighten. The lead 14 is then inserted into the heart or into a vein near the heart. After implantation of the lead 14, the stylet 112 is withdrawn and the lead 14 flexes back towards the lead's original configuration.

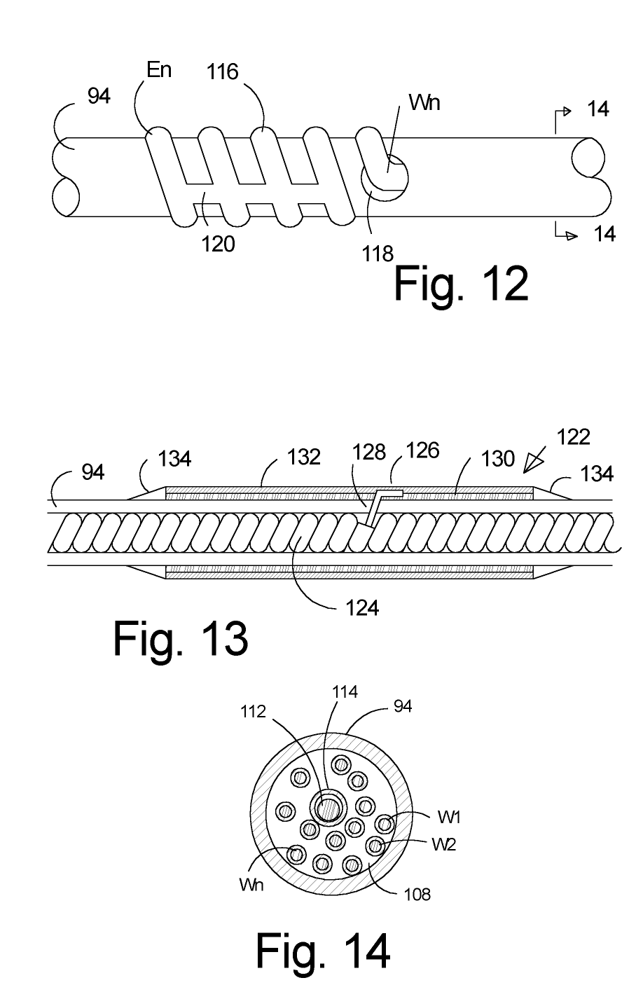

A plurality of electrodes E1, E2, E3, E4, E5, . . . En are attached to tube 94 of the lead 14. Preferably electrodes E1 . . . En are formed of coils 116 of exposed wire or cable wound about the tube 94, as shown in FIG. 12. The wire Wn passes through a predrilled hole 118 in the tube 94. The predrilled hole 118 determines the exact location of the electrode. By changing the position and spacing of the hole, leads may be designed to cluster more electrodes along a selected segment of the lead. Since the electrodes fully circumvent the tube 94, it is likely that at least some part of the electrode will be adjacent the cardiac wall. Moreover, circumferential electrodes are unlikely to perforate the heart. Preferably the coil 116 and wire Wn are formed of one continuous wire. The loops of the coil 116 are welded 120 or otherwise connected together to provide additional structural stability. Each electrode is connected to corresponding wires W1, W2, W3 . . . Wn which extend through the length of tube 94 and which are shown exiting through end 102 for the sake of clarity. Wires W1, W2, W3 . . . Wn are insulated, so that they are not shorted to each other within the tube 94. The lead 14 is more particularly disclosed in co-pending commonly assigned U.S. application Ser. No. 09/245,246 filed Feb. 5,1999, and incorporated herein by reference. Preferably the end of tube 94 and the ends of wires W1, W2, W3, etc. are coupled to a connector 104 for attaching the lead 14 to the cardiac stimulator 12. The connector 104 may have a plurality of pins Pi. Each wire W1 . . . Wn is associated with a pin.

An alternative configuration for an electrode 122 is illustrated in FIG. 13. In this configuration, a multi-filar coil 124 comprises as many insulated-wire coils as there are electrodes on the lead. The multi-filar coil 124 lies within the tube 94. At a location of an electrode 122, an end 126 of one of the wires is passed through a hole 128 in the tube 94 and laid on an inner ring 130. A hole may also be provided in the inner ring for the wire or two inner rings may be used, one ring on either side of the wire. An outer ring 132 is placed over the inner ring or rings and crimped, capturing the end 126 of the wire between the inner and outer rings. The electrical and mechanical connection between the rings and the wire may also be improved by welding or other methods. A circumferential bead 134 of glue may seal the ends of the rings and reduce sharp edges.

In addition to spiral coil or ring electrodes E1 . . . En, a distal tip electrode Ed may also be provided. The distal tip electrode Ed may also have an active fixation mechanism, for example a helical screw or tines, to secure the lead to the interior wall of the heart.

Numerous other modifications may be made to this invention without departing from its scope as defined in the attached claims.

1. An implantable cardiac stimulator comprising
a control circuit,
an output circuit controlled by said control circuit and adapted to be connected to at least one electrode implanted near the heart,
at least one sense amplifier comprising an operational amplifier in electrical communication with said control circuit through a plurality of outputs and having a plurality of inputs, each of said inputs being adapted to be connected to one of a plurality of electrodes,
said sense amplifier having a plurality of double throw switches, each of said double throw switches being connected to select an input in one position and to connect to an electrical ground in a second position.

2. The implantable cardiac stimulator of claim 1 wherein said double throw switches select an input by connecting an input to said operational amplifier.

3. The implantable cardiac stimulator of claim 2 further comprising a plurality of feedback capacitors connected between an output of said operational amplifier and an inverting input of said operational amplifier and a plurality of feedback capacitor switches, each of said feedback capacitor switches being in series with a feedback capacitor.

4. The implantable cardiac stimulator of claim 3 further comprising a plurality of feedback resistors each of said resistors being in parallel with one of said feedback capacitors and one of said feedback capacitor switches.

5. The implantable cardiac stimulator of claim 4 further comprising a plurality of feedback selection switches, each one of said feedback selection switches being connected in series between the output of said operational amplifier and one of said feedback capacitors and between the output of said operational amplifier and said feedback resistor which is in parallel with said one of said feedback capacitors.

6. The implantable cardiac stimulator of claim 5 further comprising a plurality of output switches, one side of each of said output switches being connected between the output of said operational amplifier and one of said feedback capacitors and a second side of said output switch being connected to one of said plurality of outputs.

7. The implantable cardiac stimulator of claim 6 wherein said one side of each of said output switches is further connected between a feedback resistor and a feedback capacitor switch.

8. The implantable cardiac stimulator of claim 7 wherein said one side of each of said output switches is further connected between one of said feedback selection switches and one of said feedback capacitors.

9. The implantable cardiac stimulator of claim 4 further comprising a plurality of output switches, one side of each of said output switches being connected between the output of said operational amplifier and one of said feedback capacitors and a second side of said output switch being connected to one of said plurality of outputs.

10. The implantable cardiac stimulator of claim 9 further comprising a plurality of resistors, one end of each one of said resistors being connected between said second side of an output switch and an output and a second end of said of each one of said resistors being connected to ground.

11. The implantable cardiac stimulator of claim 1 wherein said double throw switches select an input by connecting an output to an output of said operational amplifier.

12. The implantable cardiac stimulator of claim 11 further comprising a plurality of feedback capacitors connected between an output of said operational amplifier and an inverting input of said operational amplifier and a plurality of feedback resistors each of said resistors being in parallel with one of said feedback capacitors.

13. The implantable cardiac stimulator of claim 12 further comprising a plurality of feedback selection switches, each one of said feedback selection switches being connected in series between the input of said operational amplifier and one of said feedback capacitors and between the input of said operational amplifier and said feedback resistor which is in parallel with said one of said feedback capacitors.

14. The implantable cardiac stimulator of claim 13 further comprising a plurality of output switches, one side of each of said output switches being connected between the output of said operational amplifier and one of said feedback capacitors and a second side of said output switch being connected to one of said plurality of outputs.

15. The implantable cardiac stimulator of claim 14 wherein said one side of each of said output switches is further connected between said one of said feedback capacitors and one of said double throw switches.

16. The implantable cardiac stimulator of claim 15 further comprising a plurality of resistors, one end of each one of said resistors being connected between said second side of an output switch and an output and a second end of said of each one of said resistors being connected to ground.

17. The implantable cardiac stimulator of claim 12 further comprising a plurality of output switches, one side of each of said output switches being connected between the output of said operational amplifier and one of said feedback capacitors and a second side of said output switch being connected to one of said plurality of outputs.

18. The implantable cardiac stimulator of claim 17 wherein said one side of each of said output switches is further connected between said one of said feedback capacitors and one of said double throw switches.

19. The implantable cardiac stimulator of claim 18 further comprising a plurality of resistors, one end of each one of said resistors being connected between said second side of an output switch and an output and a second end of said of each one of said resistors being connected to ground.

20. The implantable cardiac stimulator of claim 1 further comprising a plurality of sense amplifiers, each of said sense amplifiers in electrical communication with said control circuit through a plurality of outputs and each of said sense amplifiers having a plurality of inputs, each of said inputs being adapted to be connected to one of a plurality of electrodes.

21. The implantable cardiac stimulator of claim 20 further comprising a lead having a plurality of electrodes, each of said electrodes being connected to an input of said sense amplifiers.

22. The implantable cardiac stimulator of claim 1 further comprising a lead having a plurality of electrodes, each of said electrodes being connected to an input of said sense amplifier.

23. An implantable medical device comprising
an electrical ground,
a plurality of electrodes,
a control circuit,
at least one sense amplifier comprising an operational amplifier in electrical communication with said control circuit through a plurality of outputs and having a plurality of inputs, each of said inputs being adapted to be connected to one of a plurality of electrodes,
said sense amplifier having a plurality of double throw switches, each of said double throw switches being connected to select an input in one position and to connect to electrical ground in a second position.

24. An implantable medical device comprising
an electrical ground,
a plurality of electrodes,
a control circuit,
at least one sense amplifier comprising an operational amplifier in electrical communication with said control circuit and having a plurality of inputs, each of said inputs being adapted to be connected to one of a plurality of electrodes, and
means for connecting to a selected input to said sense amplifier or to connect said selected input to ground.
